(19) United States
(12) Patent Application Publication
(10) Pub. No.: US 2023/0003684 A1
(43) Pub. Date: Jan. 5, 2023

(54) IS-FET NITRATE SENSOR AND METHOD OF USE

(71) Applicant: Brewer Science, Inc., Rolla, MO (US)

(72) Inventors: Jonathan J. Fury, Springfield, MO (US); Xi Cao, Tracy, CA (US); Christopher Landorf, Springfield, MO (US); Zun Chen, Springfield, MO (US); Alec Neeson, Springfield, MO (US); Weston Bowen, Springfield, MO (US); Austin Peters, Springfield, MO (US)

(21) Appl. No.: 17/856,932

(22) Filed: Jul. 1, 2022

Related U.S. Application Data
(60) Provisional application No. 63/307,432, filed on Feb. 7, 2022.
(60) Provisional application No. 63/217,339, filed on Jul. 1, 2021.

(51) Int. Cl. G01N 27/414; G01N 33/18; G01N 27/02

(52) U.S. Cl. CPC G01N 27/4148; G01N 27/4146; G01N 33/18; G01N 27/02

(57) ABSTRACT

A carbon nanotube (CNT) ion-selective field effect transistor (IS-FET) integrated device is used to detect nitrate ion in water. The device is operated as an IS-FET sensor, holding the measured potential between the drain electrode and an external reference electrode constant with a potentiometric circuit. Transduction occurs by changes in the effective CNT film gate potential with changes in the phase boundary potential of an ion-selective membrane (ISM) film. Moreover, the nitrate ISM film makes the device highly selective towards nitrate sensing. This printable IS-FET nitrate sensor enables real-time and high-resolution measurements and recording of nitrate ion in water at low cost.

### RELATED APPLICATIONS

The present application claims the priority benefit of U.S. Provisional Patent Application Ser. No. 63/217,339, filed Jul. 1, 2021, entitled PRINTED ELECTRONIC NANO-CARBON BASED DEVICES AND SYSTEMS TO IMPROVE REAL-TIME SURFACE WATER CONTAMINATION SENSING, and U.S. Provisional Patent Application Ser. No. 63/307,432, filed Feb. 7, 2022, entitled IS-FET NITRATE SENSOR AND METHOD OF USE, each of which is incorporated by reference in their entireties.

### STATEMENT REGARDING FEDERALLY SPONSORED RESEARCH OR DEVELOPMENT

This invention was made with Government support under W912HZ-18-2-0003 entitled “PRINTED ELECTRONIC NANO CARBON-BASED DEVICES AND SYSTEMS TO IMPROVE REAL-TIME SURFACE WATER CONTAMINATION SENSING,” subaward 18004-001, and under W912HZ-21-2-0019 entitled “QUANTITATIVE WATER SENSING ARRAY FOR RAPID SENSING AND CONTINUOUS MONITORING,” subaward 20206-001, both awarded by the Department of the Army ERDC. The United States Government has certain rights in the invention.

### BACKGROUND

### Field

The present disclosure relates to a sensor and method for detecting nitrates in water.

### Description of Related Art

Water contamination continues to be a major problem all over the world, and it is crucial to monitor contaminating ions in order to keep drinking water safe. One source of water contamination, nitrate ions, is widely found in bodies of water due to the excessive use of agricultural fertilizers and the discharge of wastewater from living and other industrial activities. The presence of high concentrations of nitrate ions in water may damage aquatic organisms as well as human health. The United States Environmental Protection Agency (EPA) has regulated the nitrate concentration in drinking water to 10 ppm. Therefore, there is a need for sensors that can detect nitrate ions at these low concentrations in drinking water, natural surface water, and domestic and industrial wastewater.

### SUMMARY

The present disclosure provides a sensor comprising a substrate, a source electrode on the substrate, a drain electrode on the substrate, a carbon nanotube gating layer connecting the source electrode and the drain electrode, an ion selective membrane on the carbon nanotube gating layer, and a counter electrode on the substrate. There is no direct physical contact between the counter electrode and any of the source electrode, drain electrode, carbon nanotube gating layer, or ion selective membrane. The ion selective membrane comprises:

a polymer, an epoxyacrylate oligomer, or both a polymer and an epoxyacrylate oligomer, the polymer being chosen from polyvinyl chloride, polyacrylate, polymethacrylate, or combinations thereof;

an ionophore chosen from cyanoaqua-cobyrinic acid heptakis(2-phenylethyl ester), 1,6,10,15-tetraoxa-2,5,11,14-tetraaza-cyclooctodecane, 1,7,11,17-tetraoxa-2,6,12,16-tetraazacycloe-icosane, 9,11,20,22-tetrahydrotetrabenzo[d.f,k,m] [1,3,8,10]tetra-azacyclotetradecine-10,21-dithione, 9-hexadecyl-1,7,11,17-tetraoxa-2,6,12,16-tetraazacycloeicosane, or combinations thereof;

an ion exchanger chosen from tridodecylmethyl ammonium nitrate, tetradodecyl ammonium nitrate, tetraoctylammonium nitrate, potassium tetrakis(4-chlorophenyl) borate, tetrakis(4-chlorophenyl)borate tetradodecylammonium salt, or combinations thereof; and

a plasticizer.

In another embodiment, a sensing device comprising the above sensor is provided. The sensing device further comprises a reference electrode and a power source, wherein the reference electrode and power source are connected to the sensor.

In another embodiment, the disclosure provides a method of monitoring for the presence of an analyte in water, where the method comprises contacting the sensor with water to be monitored.

### BRIEF DESCRIPTION OF THE DRAWINGS

FIG. 1 is a is a schematic (not to scale) of one embodiment of an IS-FET nitrate sensor fabrication process;

FIG. 2 is a top view of a schematic (not to scale) of one embodiment of an IS-FET nitrate sensor as described herein with electrical connections and reference electrode;

FIG. 3 is a fragmentary sectional view of the IS-FET nitrate sensor of FIG. 2 taken along lines 3-3;

FIG. 4 is a photograph of one embodiment of a Ag/AgCl reference electrode suitable for use with the IS-FET nitrate sensors disclosed herein;

FIG. 5 is a block diagram of a sensor circuit of one embodiment according to the disclosure herein;

FIG. 6 is another block diagram of a sensor circuit of a second embodiment according to the disclosure herein;

FIG. 7 is a schematic depiction of a nitrate sensing system that includes a nitrate sensor as described herein;

FIG. 8 is a CNT film gate response of a non-enriched (i.e., no ion selective membrane) CNT film in 1 mM potassium nitrate+0.1% ammonium sulfate supporting electrolyte (Example 4);

FIG. 9 is a solution matrix setup for the IS-FET sensitivity testing described in Example 5;

FIG. 10 depicts graphs of drain current (Id) vs. time step response of ISFET device in several solutions of nitrate (left) and average of each step response vs. logarithm of nitrate concentration (right), both generated as described in Example 5;

FIG. 11 shows a solution matrix setup for IS-FET selectivity testing as described in Example 6;

FIG. 12 provides graphs of step-response in selectivity tests of nitrate devices in the presence of various interfering ions, with these graphs being generated as described in Example 6; and

FIG. 13 is a graph of the results of a drifting test carried out for about 25 days as described in Example 7.

### DETAILED DESCRIPTION

The present disclosure is concerned with ion-selective field effect transistor (“IS-FET”) sensors, and systems and methods that utilize those sensors to detect and/or measure nitrates in water, preferably in a substantially continuous or ongoing intermittent manner.

### Sensors

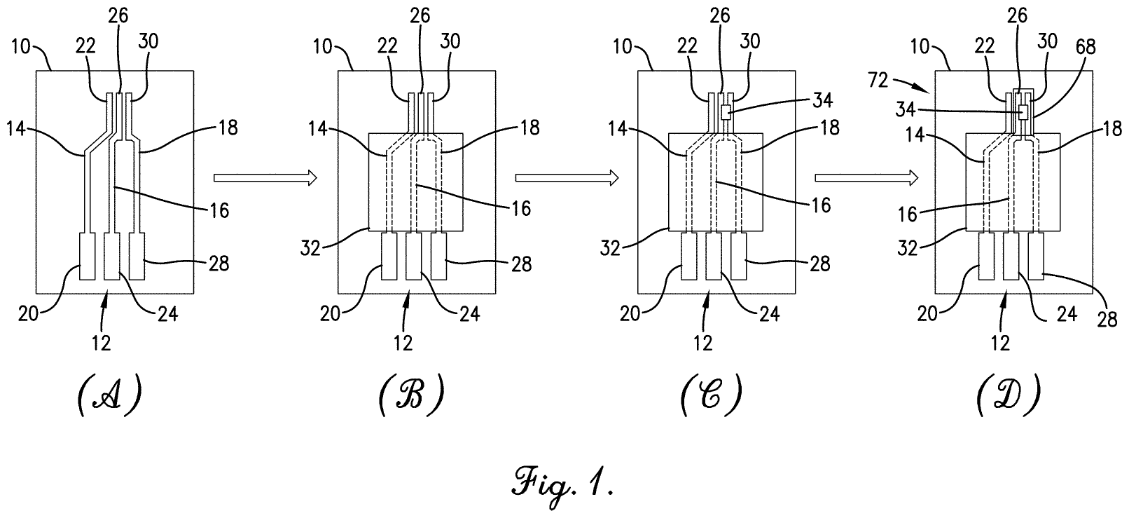

Referring to FIGS. 1(A)-(D), an exemplary sensor formation process is described.

### FIG. 1(A)

### Substrate and Electrodes

FIG. 1(A) shows a substrate 10 having an electrode system 12 deposited thereon.

### 1. Substrate 10

Substrate 10 may be formed from any number of materials, including those selected from the group consisting of polymers, ceramics, metals, crystalline silicon, and combinations thereof. Suitable organic polymers include those selected from the group consisting of cyclic olefin polymers (such as those sold as films under the name Zeonor® by Zeon Corporation, with ZEONEX® ZF14-188 being one preferred such film), fluorinated polymers such as polytetrafluoroethylene (“PTFE,” such as those sold as films under the name Teflon® by DuPont), copolymers of tetrafluoroethylene and hexafluoropropylene (“FEP” and “PFA”), polyvinylidene fluoride, polyether ether ketone (“PEEK”), polyetherimide polyphenylene sulfide, polysulfones, polyoxymethylene (“POM”), polyimides, polyamides, polyether sulfones, polyethylene terephthalate (“PET”), polyacrylates, polymethacrylates, polystyrenes, polyesters, polyethylene naphthalate, and combinations of the foregoing. Suitable ceramics include alumina and aluminum nitride.

The substrate 10 preferably has a low water absorbency and low moisture permeability. Preferably, the water absorbency is less than about 2%, more preferably less than about 1%, and even more preferably about 0.1% to about 0.5% according to ASTM method D570. It is also preferred that the substrate 10 does not experience hygroscopic expansion or similar deformation, which can generally be determined visually.

The substrate 10 preferably has a surface energy of from about 25 dynes/cm to about 50 dynes/cm, more preferably from about 28 dynes/cm to about 35 dynes/cm as determined by a contact angle meter. The substrate 10 should have a low electronic conductivity, preferably less than about 0.01 μS/cm, more preferably less than about 0.001 μS/cm, and even more preferably about 0 μS/cm as determined by a multimeter or four point probe. The substrate 10 has a low ionic conductivity, preferably less than about 0.01 μS/cm, more preferably less than about 0.001 μS/cm, and even more preferably about 0 μS/cm as determined by electrochemical impedance spectroscopy of the substrate material. Surface treatments such as UVO or plasma treatments, may be used to improve adhesion of printed layers to the substrate 10, if desired.

The substrate 10 is preferably planar, or at least presents a substantially planar surface to facilitate electrode system 12 deposition using methods described below. Substrate 10 is generally rectangular in shape, but could also be configured to be square, circular, etc., as may be desired for the particular application. Substrate 10 is preferably sized such that the entire electrode system 12 can fit on the surface of substrate 10 and within the outer perimeter of substrate 10. In one embodiment, the substrate 10 is preferably about 5 mm to about 10 mm wide, more preferably about 7 mm to about 9 mm wide, and even more preferably about 8 mm wide and/or preferably about 20 mm to about 25 mm long, more preferably about 21 mm to about 24 mm long, and even more preferably about 23 mm long. Regardless of the size or shape, the average thickness (as measured by an ellipsometer) of substrate 10 is generally about 50 μm to about 5 mm, preferably about 50 μm to about 2.5 mm, more preferably about 75 μm to about 1,000 μm, and even more preferably about 100 μm to about 300 μm.

It will be appreciated that, in some embodiments, multiple devices may be formed on a single substrate 10 (sized and shaped for this purpose) and then diced or otherwise separated into individual substrates 10 of the aforementioned dimensions.

### 2. Electrode System 12

Still referring to FIG. 1(A), the electrode system 12 comprises a counter electrode 14, a drain electrode 16, and a source electrode 18. Counter electrode 14 has a counter lead end 20 and a counter working end 22. Similarly, drain electrode 16 has a drain lead end 24 and a drain working end 26, while source electrode 18 has a source lead end 28 and source working end 30.

Counter electrode 14, drain electrode 16, and source electrode 18 are preferably substantially planar and/or could be interdigitated electrodes. In another embodiment, counter electrode 14, drain electrode 16, and source electrode 18 comprise non-interdigitated electrodes. The material from which electrodes 14, 16, and 18 are formed is chosen so that the electrodes 14, 16, and 18 exhibit high conductivity. That is, it is preferred that the electrodes 14, 16, and 18 have a total equivalent series resistance as measured by a four point probe or a multimeter of less than about 5Ω, preferably less than about 3Ω, and more preferably less than about 1Ω, preferably at about 10° C. to about 30° C.

Suitable materials for forming counter electrode 14, drain electrode 16, and source electrode 18 include those chosen from silver, gold, platinum, conductive polymers (e.g., poly(3,4-ethylenedioxythiophene-poly(styrene sulfonate), polyaniline), doped silicon, conductive carbon nanotubes (CNTs), amorphous carbon, graphite, graphene, carbon nanobuds, glassy carbon, carbon nanofibers, palladium, copper, aluminum, nickel, CNT/graphene-conductive polymer composites, and combinations thereof. Counter electrode 14, drain electrode 16, and source electrode 18 can be formed from the same material or different materials, depending on the designer's preference.

Each of counter electrode 14, drain electrode 16, and source electrode 18 is preferably about 200 μm to about 2 cm wide at their respective working ends 22, 26, 30, more preferably about 400 μm to about 800 μm wide at their respective working ends 22, 26, 30, and even more preferably about 500 μm wide at their respective working ends 22, 26, 30. In some embodiments, each of counter electrode 14, drain electrode 16, and source electrode 18 has an overall length of about 10 mm to about 50 mm, more preferably about 15 mm to about 30 mm, and even more preferably about 19.5 mm. Finally, in some embodiments, each of counter electrode 14, drain electrode 16, and source electrode 18 has an average thickness (as measured by an ellipsometer) of about 500 Å to about 2,000 Å, preferably about 600 Å to about 2,000 Å, more preferably about 800 Å to about 1,500 Å, and even more preferably about 1,000 Å. It will be appreciated that each of counter electrode 14, drain electrode 16, and source electrode 18 can have the same or different average thicknesses, widths, and/or overall lengths. In one embodiment, the counter electrode 14 has a surface area equal to or greater than the surface area of the source electrode (18) and/or drain electrode (16).

The electrode system 12 may be deposited by any appropriate method, including sputtering, electron beam evaporation, ion-assisted electron beam evaporation, thermal evaporation, ink-jet printing, screen printing, gravure printing, or flexography.

### FIG. 1(B)

### Encapsulant Layer

Referring to FIG. 1(B), an encapsulant layer 32 is suitably formed over all areas of counter electrode 14, drain electrode 16, and source electrode 18, except for areas at the working ends 22, 26, 30 that are to be exposed to the analyte and areas at the lead ends 20, 24, 28 that are to be used for making electrical connections to the electrode system 12. More particularly, encapsulant layer 32, which is preferably planar, is formed so it extends substantially continuously over areas of the electrodes 14, 16, 18 intermediate to lead ends 10, 24, 28 and respective working ends 22, 26, 30, thus protecting those intermediate areas from analyte contact. In the illustrated embodiment, encapsulant layer 32 is generally rectangular in shape, although that shape can be altered depending on the areas to be protected from analyte contact. Additionally, the encapsulant layer 32 is typically in contact with portions of substrate 10 around and between counter electrode 14, drain electrode 16, and source electrode 18 at those areas intermediate to lead ends 10, 24, 28 and respective working ends 22, 26, 30.

The encapsulant layer 32 should be a dielectric material and preferably has an ionic impedance (measured by electrochemical impedance spectroscopy) of at least about 1 MΩ, preferably at least about 5 MΩ, and more preferably at least about 10 MΩ. The encapsulant layer 32 should have a resistance of at least about 1 MΩ, preferably at least about 5 MΩ, and more preferably at least about 10 MΩ. The encapsulant layer 32 should be water resistant and exhibit sufficient adhesion to substrate 10 and electrode system 12 to prevent leakage and/or diffusion of the analyte solution around and/or through the encapsulant layer 32.

The encapsulant layer 32 can be formed from a material chosen from one or more of cyclic olefin polymers (such as those sold as films under the names Zeonor® and Zeonex® by Zeon Corporation, with Zeonex® ZF14-188 and Zeonor® 790R being two preferred such films), fluorinated polymers such as polytetrafluoroethylene, copolymers of tetrafluoroethylene and hexafluoropropylene, polyvinylidene fluoride, polyether ether ketone, polyetherimide polyphenylene sulfide, polysulfones, polyoxymethylene, polyimides, polyamides, polyether sulfones, polyethylene terephthalate, polyacrylates, polymethacrylates, polystyrenes, polyesters, polyethylene naphthalate, polysilicones, and combinations of the foregoing. In one embodiment, the encapsulant is DuPont 5018 dielectric material. In one embodiment, the material from which encapsulant layer 32 is formed is the same as the material from which substrate 10 is formed.

The encapsulant layer 32 may be deposited by any appropriate means, including screen printing, spray coating, Aerosol Jet® printing, inkjet printing, dip coating, airbrush techniques, flexographic printing, gravure printing, lithographic techniques, spin coating, or lamination. An additional UV cure or baking step may be used to cure the encapsulant layer 32.

The encapsulant layer 32 is preferably about 3 mm to about 15 mm wide, more preferably about 5 mm to about 10 mm side, and even more preferably about 7 mm wide, and/or about 5 mm to about 20 mm long, more preferably about 8 mm to about 15 mm long, and even more preferably about 12 mm long. The average thickness (as measured with an ellipsometer) of the encapsulant layer 32 is preferably about 0.01 μm to about 10 μm, more preferably about 0.1 μm to about 5 μm, and even more preferably about 1 μm to about 5 μm.

### FIG. 1(C)

### CNT Gating Layer

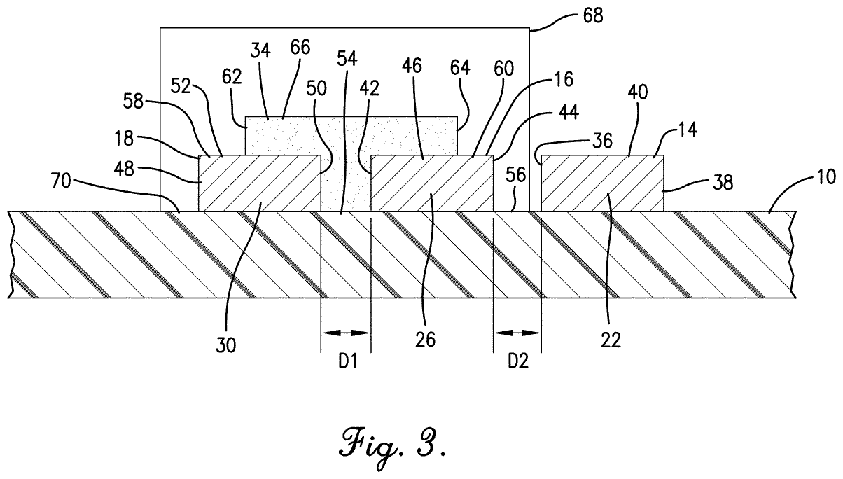

Referring to FIG. 1(C), a carbon nanotube (“CNT”) gating layer 34 is formed on and between drain electrode 16 and source electrode 18 at their working ends 26, 30. More specifically and referring to FIG. 3, which shows a sectional view through the working ends 22, 26, 30, and CNT gating layer 34, counter electrode 14 comprises a first counter electrode sidewall 36 facing generally toward drain electrode 16 and a second counter electrode sidewall 38 facing generally away from drain electrode 16. First counter electrode sidewall 36 is connected to second counter electrode sidewall 38 by upper counter electrode surface 40 extending between the sidewalls 36, 38. The first counter electrode sidewall 36 is spaced closer to the drain electrode 16 than is the second counter electrode sidewall 38.

Drain electrode 16 comprises a first drain electrode sidewall 42 facing generally toward source electrode 18 and facing generally away from counter electrode 14. Drain electrode 16 further comprises a second drain electrode sidewall 44 facing generally toward counter electrode 14 and facing generally away from source electrode 18. First drain electrode sidewall 42 is connected to second drain electrode sidewall 44 by upper drain electrode surface 46 extending between the sidewalls 42, 44. The first drain electrode sidewall 42 is spaced closer to the source electrode 18 than is the second drain electrode sidewall 44. The second drain electrode sidewall 44 is spaced closer to the counter electrode 14 than is the first drain electrode sidewall 42.

Source electrode 18 comprises a first source electrode sidewall 48 facing generally away from drain electrode 16 and a second source electrode sidewall 50 facing generally towards drain electrode 16. First source electrode sidewall 48 is connected to second source electrode sidewall 50 by upper source electrode surface 52 extending between the sidewalls 48, 50. The first source electrode sidewall 48 is spaced farther from the drain electrode 16 than is the second source electrode sidewall 50.

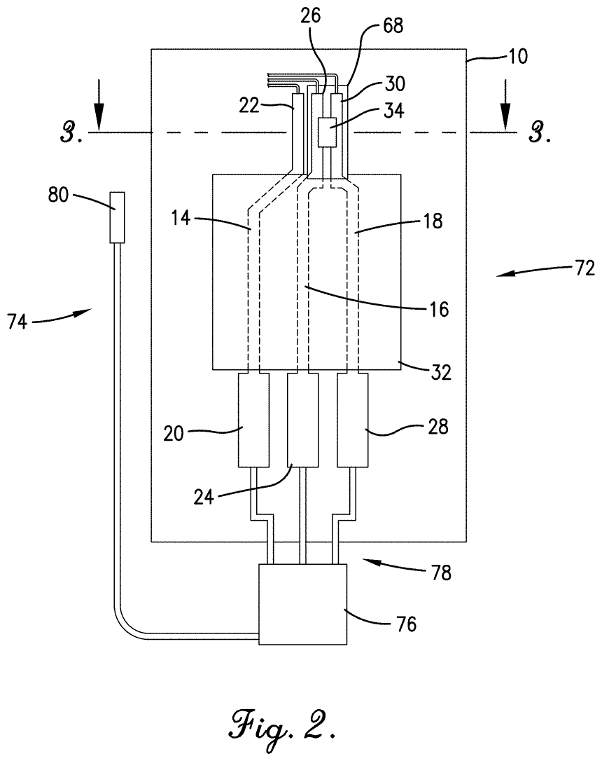

Drain electrode 16 and source electrode 18 are spaced apart at their working ends 26, 30, respectively (FIG. 2), such that there is a distance “D1” (FIG. 3) between first drain electrode sidewall 42 and second source electrode sidewall 50, creating first exposed substrate portion 54. Distance D1 is preferably about 100 μm to about 1 cm, more preferably about 400 μm to about 800 μm, and even more preferably about 500 μm. Similarly, drain electrode 16 and counter electrode 14 are spaced apart at their working ends 26, 22 such that there is a distance “D2” between second drain electrode sidewall 44 and first counter electrode sidewall 36, creating second exposed substrate portion 56. Distance D2 is preferably about 200 μm to about 2 cm, more preferably about 400 μm to about 1000 μm, and even more preferably about 500 μm. “Exposed” as used in this context means that there is not any electrode material on the surface of substrate 10 in these areas, although it is noted other materials may be on the substrate 10 at its exposed areas 54, 56. Distance D1 is preferably equal to or less than distance D2.

CNT gating layer 34 is applied so as to contact some, or even all, of upper source electrode surface 52 and upper drain electrode surface 46, leaving uncovered upper source electrode surface 58 and uncovered upper drain electrode surface 60, thus creating contact between drain electrode 16 and source electrode 18 through the CNT gating layer 34. The material forming CNT gating layer 34 is further applied so that it is deposited between drain electrode 16 and source electrode 18, so that CNT gating layer 34 is on first exposed substrate portion 54 and in contact with first drain electrode sidewall 42 and second source electrode sidewall 50, filling the spacing D1.

CNT gating layer 34 has a first sidewall 62 that extends away from upper source electrode surface 52. CNT gating layer 34 further has a second sidewall 64 that extends away from upper drain electrode surface 46. CNT first sidewall 62 and second sidewall 64 are joined by upper CNT surface 66, which is suitably substantially planar in nature and extends between the sidewalls 62, 64. CNT gating layer 34 does not contact any part of counter electrode 14.

Referring again to FIG. 1(A), it will be appreciated that the spatial relations, including, but not limited to D1 and D2, the relative positions of the CNT gating layer 34, electrodes 14, 16, 18, and the portions thereof described in this “CNT Gating Layer” section are applicable at the working ends 22, 26, 30 of the electrodes. Further, as illustrated in FIG. 1(A), the spacings between the electrodes 14, 16, 18, and their orientations relative to one another, suitably change along the length of the electrode system 12. For example, sections of the electrodes 14, 16, 18 diverge from one another moving from the working ends 22, 26, 30 toward the lead ends thereof 20, 24, 28, thereby altering the distances between the electrodes from D1 and D2. Also, short sections of the electrode sidewalls are suitably parallel to one another at the lead ends 20, 24, 28 due to the rectangular-shaped lead ends in the embodiment depicted in FIG. 1(A).

CNT gating layer 34 preferably has an average thickness (as measured by an ellipsometer) of about 1 nm to about 1,000 nm, more preferably about 1 nm to about 400 nm, and even more preferably about 50 nm to about 300 nm. In one embodiment, this thickness is the average thickness of the CNT gating layer 34 on upper drain electrode surface 46 and/or upper source electrode surface 52. In another embodiment, this thickness is the average thickness of the CNT gating layer 34 measured at first exposed substrate portion 54.

The width of the CNT gating layer 34 is preferably about 100 μm to about 20 mm, and more preferably about 0.5 mm to about 5 mm. The length of the CNT gating layer 34, which is more easily seen in FIG. 1 or 2, is preferably from about 0.001 mm to 20 mm, and more preferably from about 0.5 mm to 5 mm. In one embodiment, the CNT gating layer 34 covers part of each of the source and drain electrodes 18, 16. In another embodiment, the CNT gating layer 34 covers all of each of the source and drain electrodes 18, 16.

CNT gating layer 34 is formed from a conductive carbon nanotube dispersion having carbon nanotubes, a solvent, and optionally a surfactant. Suitable carbon nanotubes comprise semiconductive carbon nanotubes, metallic carbon nanotubes, multi-walled carbon nanotubes, single-walled carbon nanotubes, and mixtures of the foregoing. The carbon nanotubes may be nonfunctionalized, or may be functionalized with groups such as pyrene groups, carboxylic acid groups, sulfonic acid groups, amine groups, and combinations thereof.

Suitable solvents or dispersants comprise water, alcohols (e.g., isopropanol), diols (e.g., 1,2-propanediol, 2-methyl-1,3-propanediol), and polar water-miscible solvents (e.g., acetone, n-methyl-2-pyrrolidone, 1,3-dimethyl-2-imidazolidinone), and combinations thereof.

Suitable surfactants comprise sodium dodecylbenzenesulfonate, sodium cholate, polyoxyethylene octyl phenyl ether (such as that sold under the name Triton™ X-100, by Dow Chemical Company), polyoxyethylene sorbitol ester (sold as Tween® 20 or Tween® 80, by Sigma Aldrich), sodium dodecyl sulfate, and mixtures thereof.

The CNT dispersions used to form CNT gating layer 34 preferably comprise about 0.00001% to about 1% by weight CNTs, more preferably about 0.00001% to about 0.01% by weight CNTs, and even more preferably from about 0.00001% to about 0.00005% by weight CNTs. The amount of solvent in the CNT dispersion is preferably about 89% to about 99.9999% by weight, more preferably about 99% to about 99.9999% by weight, and even more preferably about 99.9995% to about 99.9999% by weight. When a surfactant is used, the amount of surfactant in the CNT dispersion is preferably about 0.001% to about 10% by weight, more preferably about 0.1% to about 6% by weight, and even more preferably about 0.3% to about 1% by weight. These percentages by weight are based upon the total weight of the CNT dispersion taken as 100% by weight.

The CNT dispersions used to form CNT gating layer 34 can be formed by dispersing the carbon nanotubes in the solvent, optionally in the presence of a surfactant, to form a substantially homogeneous dispersion. Preferred methods of mixing or dispersing include probe ultrasonicator, bath sonicator, microfluidic system, planetary mixer, and/or 3-roll mill.

The CNT dispersions may be deposited by any suitable technique, including screen printing, spray coating, Aerosol Jet® printing, inkjet printing, dip coating, airbrush techniques, flexographic printing, gravure printing, lithographic techniques, and spin coating. After deposition, additional wash steps may be performed to remove surfactant from the CNT gating layer 34, and/or to remove or alter functional groups in the CNT gating layer 34, depending on the user's needs.

CNT gating layer 34 provides the sensing component in that its electronic properties respond directly to a change in the ion selective membrane (discussed below), including changes in its electronic structure, defect state, and/or electronic carrier density. In a traditional IS-FET design, the function of the gate electrode is mainly to respond to the input gating voltage by altering the conductivity between the drain and source. In the case of the CNT gating layer 34 in the formed IS-FET device, upon exposure to ions the electronic properties of the CNT gating layer 34 under a certain gate voltage will change proportionally to the change in the concentration of the target ion (i.e., nitrates). The change in the electronic properties will result in an output signal change of at least about 5% for every 100% change in nitrate concentration, and preferably at least about 20% for every 100% change in nitrate concentration. The resistance of the CNT gating layer 34 is preferably about 1 kΩ to about 20 kΩ, and more preferably about 5 kΩ to about 10 kΩ.

### FIG. 1(D)

### Ion-Selective Membrane

Referring to FIG. 1(D), an ion-selective membrane (“ISM”) 68 is formed on CNT gating layer 34, drain working end 26 of drain electrode 16, and source working end 30 of source electrode 18. Referring to FIG. 3, ISM 68 entirely covers and encompasses CNT gating layer 34, fully covering CNT gating layer 34's first sidewall 62, second sidewall 64, and upper surface 66. Additionally, ISM 68 fully encompasses and covers any portions of drain electrode 16 and source electrode 18 not covered by CNT gating layer 34. For example, ISM 68 suitably extends from at least the encapsulant layer 32 to at least the ends of the drain electrode 16 and source electrode 18 at their working ends 26, 30. More particularly, ISM 68 contacts and covers uncovered upper source electrode surface 58 of source electrode 18 and also uncovered upper drain electrode surface 60 of drain electrode 16. The ISM 68 suitably encapsulates the first source electrode sidewall 48 of the source electrode 18 and the second drain electrode sidewall 44 of the drain electrode 16 that faces the counter electrode 14. The ISM 68 also suitably covers and encapsulates any first exposed substrate portion 54 of the substrate 10 not covered by the CNT gating layer 34. ISM 68 also covers a portion of the substrate 10's surface at second exposed substrate portion 56 as well as a portion 70 of substrate 10's surface adjacent first source electrode sidewall 48 that was previously exposed. Notably, while ISM 68 does contact substrate 10 at second exposed substrate portion 56, it is spaced away from first counter electrode sidewall 36 and does not contact any part of counter electrode 14. In another embodiment, ISM 68 also contacts all or part of counter electrode 14.

ISM 68 is substantially planar and has an average thickness (as measured by an ellipsometer) of about 500 nm to about 10 μm, preferably about 0.5 μm to about 10 μm, and more preferably about 2 μm to about 5 μm. In one embodiment, this thickness is the average thickness of ISM 68 on upper CNT surface 66. In another embodiment, this thickness is the average thickness of ISM 68 measured at uncovered upper source electrode surface 58 and/or uncovered upper drain electrode surface 60.

ISM 68 is sized and shaped so that it covers and encompasses all of the sensing areas of drain electrode 16 and source electrode 18. In other words, ISM 68 entirely covers and encompasses all of the drain working end 26 and source working end 30 that is not covered by encapsulant layer 32 (see FIGS. 1 and 2) as well as the entirety of CNT gating layer 34 (as described above). This typically translates to a width of about 0.1 mm to about 20 mm, and more preferably about 0.5 mm to about 5 mm, and/or a length of about 0.1 mm to about 30 mm, and more preferably about 0.5 mm to about 50 mm.

ISM layer 68 is an ion selective layer, inducing analyte ion concentration transfer from the aqueous phase to the organic phase, creating an interfacial charge separation that is the origin of the interfacial phase boundary potential in the IS-FET device. To this end, ISM 68 is preferably formed from an ISM dispersion that comprises an ionophore, an ion exchanger or electrolyte, a polymer and/or oligomer, and a plasticizer dispersed or dissolved in a solvent.

Suitable ionophores comprise cyanoaqua-cobyrinic acid heptakis(2-phenylethyl ester), 1,6,10,15-tetraoxa-2,5,11,14-tetraazacyclooctodecane, 1,7,11,17-tetraoxa-2,6,12,16-tetraaza-cycloeicosane, 9,11,20,22-tetrahydrotetrabenzo[d.f,k,m] [1,3,8,10]tetraazacyclotetradecine-10,21-dithione, 9-hexadecyl-1,7,11,17-tetraoxa-2,6,12,16-tetraazacycloeicosane, and combinations thereof. The amount of ionophore is preferably from about 0.1% to about 5% by weight, more preferably about 0.5% to about 3% by weight, and even more preferably about 1.5% to about 2.5% by weight, based on the total weight of the ISM dispersion taken as 100% by weight.

Suitable ion exchangers comprise tridodecylmethyl ammonium nitrate, tetradodecyl ammonium nitrate, tetraoctylammonium nitrate, potassium tetrakis(4-chlorophenyl) borate, tetrakis(4-chlorophenyl)borate tetradodecylammonium salt, and combinations thereof. The amount of ion exchanger included is preferably about 0.1% to about 5% by weight, more preferably from about 0.5% to about 2.5%, and even more preferably from 1% to 2% by weight, based on the total weight of the ISM dispersion taken as 100% by weight.

Suitable polymers and oligomers include those that will function as a polymeric or oligomeric matrix within the dispersion and final ISM 68. Such polymers comprise polyvinyl chloride, polyacrylate, polymethacrylate, and combinations thereof. Suitable oligomers include epoxyacrylate oligomers, The total polymer and oligomer present is typically included in the ISM dispersion at levels of about 0.1% to about 60%, more preferably about 1% to about 30%, and even more preferably 3% to 10% by weight, based on the total weight of the ISM dispersion taken as 100% by weight.

Suitable plasticizers comprise 2-nitrophenyl octyl ether, dibutyl phthalate, bis(2-ethylhexyl) sebacate, bis(2-ethylhexyl) phthalate, and combinations thereof. The amount of plasticizer is preferably about 5% to about 25% by weight, more preferably about 10% to about 20%, and even more preferably from 15% to 18% by weight, based on the total weight of the ISM dispersion taken as 100% by weight.

Suitable solvents comprise cyclohexanone, acetone, tetrahydrofuran, N-methyl-2-pyrrolidone, 1,3-dimethyl-2-imidazolidinone, N,N-dimethylformamide, and combinations thereof. The amount of solvent in the ISM dispersion is preferably about 50% to about 99% by weight, more preferably about 60% to about 90% by weight, and even more preferably about 70% to about 80%, based on the total weight of the ISM dispersion taken as 100% by weight.

The ISM dispersion can be deposited to form the ISM 68 by any suitable technique, including screen printing, spray coating, Aerosol Jet® printing, inkjet printing, dip coating, airbrush techniques, flexographic printing, gravure printing, lithographic techniques, spin coating, and lamination. Regardless of the formation method, final ISM 68 comprises a polymer matrix that has the other ingredients physically suspended and dispersed within that matrix.

The process described above yields a sensor 72 (FIG. 2). Sensor 72 can be used in a sensing device 74, which is schematically depicted in FIG. 2. Sensing device 74 includes a power source 76, electrical connections 78, and a reference electrode 80. Sensor 72 is electrically connected via electrical connections 78 at counter lead end 20, drain lead end 24, and source lead end 28.

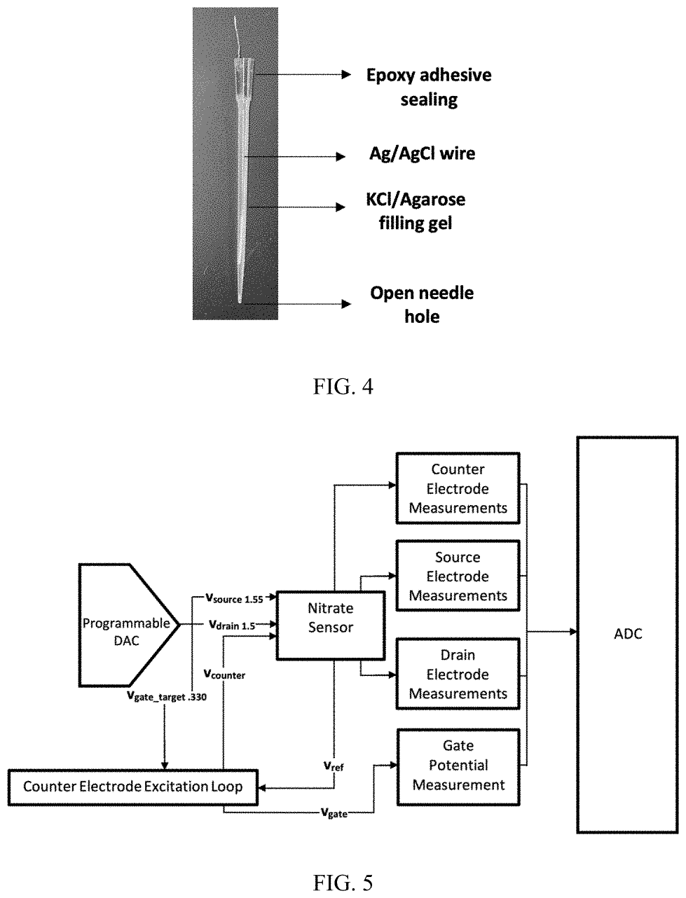

Reference electrode 80 improves the lifecycle of the sensor 72 and can be any reference electrode typically utilized in IS-FET devices. An Ag/AgCl (supersaturated potassium, KCl) reference electrode is preferred. One such preferred reference electrode may be formed of a plastic or glass tube filled with saturated KCl gel, with an Ag/AgCl wire inserted into the tube. The gel and Ag/AgCl wire are sealed from the environment, such as by an epoxy adhesive. FIG. 4 provides a photograph of one such reference electrode.

This four-electrode sensing device 74 created by the above-described components holds the measured potential between the drain electrode 16 and the reference electrode 80 constant with a potentiometric circuit, as discussed in more detail below.

### Circuitry

The electronics used to power and measure the nitrate sensor device 74 can be any that are conventionally used. In one embodiment, the electronic configuration comprises precision voltage sources, differential amplifiers, transimpedance amplifiers, and standard operational amplifiers. FIG. 5 provides a block diagram of one such embodiment of the sensor circuit.

One or more programmable precision voltage source (digital-to-analog converter, DAC) provide precision voltages needed to properly electrically bias the IS-FET junction. In one embodiment, the programmable precision voltage source provides V_{source} to the source electrode, provides V_{drain} to the drain electrode, and produces a 0.330 V reference potential on the reference electrode, V_{ref}, which is used in a counter electrode excitation loop. V_{source} is greater than V_{drain}, and both values are selected experimentally to optimize current draw to allow the current to be sufficiently large to measure accurately and precisely, while avoiding drawing too much current and potentially burning out the device 74.

In one embodiment, V_{source} is preferably about 0.05 V to about 3.0 V, and more preferably about 1.55 V. V_{drain} is preferably about 0 V to about 3.0 V, and more preferably about 1.50 V. The difference between V_{source} and Vᵣₐᵢₙ, V_{diff}, is determined experimentally, and is preferably about 1 mV to about 500 mV, more preferably about 10 mV to about 100 mV, and even more preferably about 50 mV. The source current, I_{source}, and drain current, I_{drain}, are collected as meaningful measurements.

The counter electrode excitation loop continuously calculates the difference between the V_{drain} and the V_{ref}. This calculation defines the value of the gate voltage, V_{gate}. An appropriate gate voltage reference, V_{gate target} is compared to the measured gate voltage. The optimal values of V_{gate target} are determined experimentally and are preferably about 200 mV to about 500 mV, and even more preferably about 330 mV. The difference between the programmed voltage and the measured gate voltage is then applied to the counter electrode as V_{counter}. These relationships are defined as:

V_{gate}=V_{drain}−V_{ref}

V_{counter}=V_{gate}−V_{gate target}

Power is applied to the counter electrode from a different precision source because the counter voltage is meant to change over time. An additional amplifier is used to record the real-time value of the counter voltage, and there is also preferably an additional two-stage circuit (buffer stage and trans-impedance amplifier) used to measure the current of the counter electrode.

In another embodiment, the circuitry comprises a highly configurable precision analog microcontroller (MCU) with chemical sensor interface, such as an ADuCM355 analog front end sold by Analog Devices, which features two potentiostatic circuits. Using both channels, the MCU can be programmed to provide the same electrical requirements in much less space. The components used to accomplish this include a precision DAC, potentiostatic amplifier, transimpedance amplifier, and precision ADC. All of the components are conveniently programmable and integrated into the MCU system on a chip.

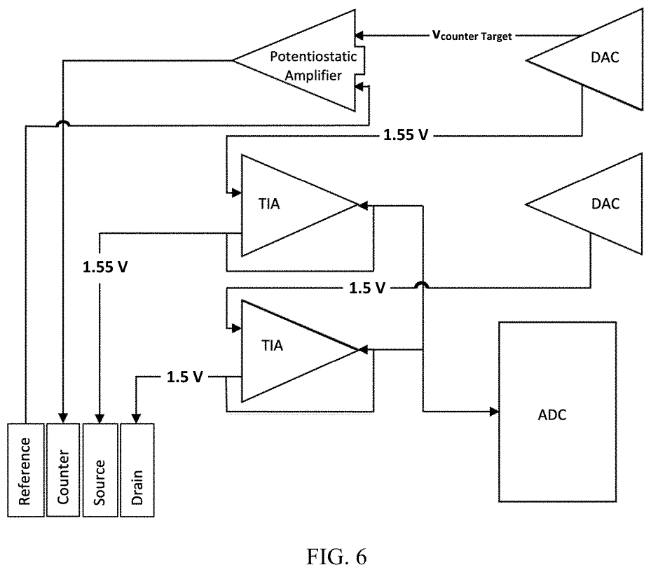

FIG. 6 schematically depicts the equivalent circuit produced from the firmware settings applied to the MCU analog front end. This circuit layout uses both of the available low power potentiostatic channels to drive and measure the IS-FET nitrate sensing device. The counter electrode target voltage is set by the following relationship:

V_{counter target}=V_{drain}−V_{gate programmed}

This approach allows the use of reference electrode feedback to actively control the output of the counter electrode and achieve the required gate voltage that has been programmed by the user.

The transimpedance amplifiers (TIAs) serve two purposes: maintenance of the set bias between the source and drain electrodes, V_{diff}, and measurement of the corresponding current on each electrode. Not pictured in the feedback loop of each TIA, there exists a precision programmable resistor that can be used to determine the current flow of the sensor electrodes. The ADC is configured to measure several points of interest along the circuit paths so that every relevant metric can be directly measured or expressed from a combination of other measurements.

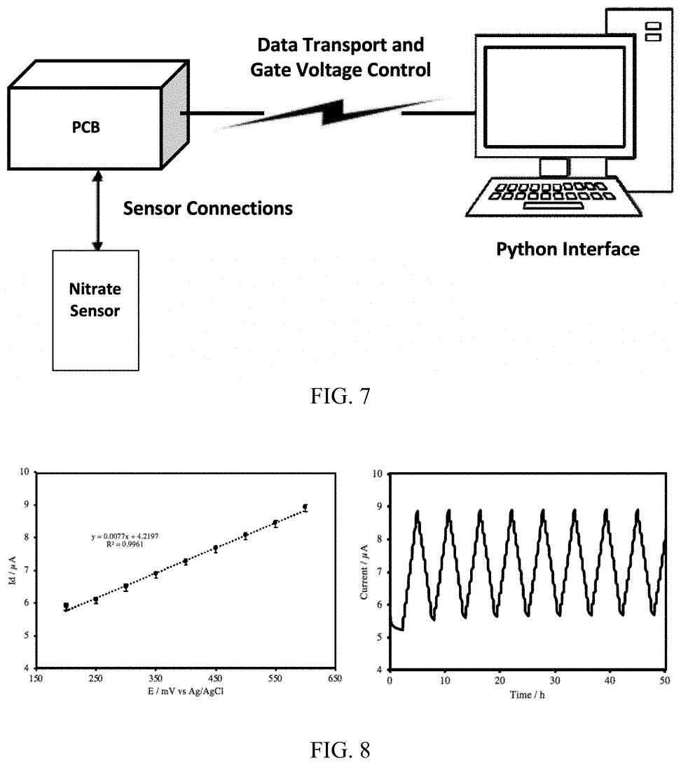

In either embodiment, the output of the ADC is configured to communicate with a computer that is programmed to correlate the signals to nitrate ion concentration, display the data to a user, and store the data. The computer may also be configured to control the inputs to the circuit controlling and reading the IS-FET nitrate sensing device. FIG. 7 schematically depicts a diagram of such a system comprising the IS-FET nitrate sensing device, a printed circuit board, and computer.

### Method of Use

In use, the source, drain, counter, and reference electrodes 12, 16, 18, 80 of the IS-FET nitrate-sensing device 74 are exposed to the analyte of interest. As nitrate ions permeate the ISM 68, the electrical properties of the CNT gating layer 34 are changed. The changed electrical properties include conductivity, resistance, impedance, thermoelectricity, temperature coefficient of resistance, and combinations thereof. Preferably, the change in the electrical properties of the CNT gating layer 34 is proportional to the change in concentration of nitrate ions in the analyte. In one embodiment, the impedance of the CNT gating layer 34 decreases with increasing nitrate ion concentration, which in turn causes the current across the CNT gating layer 34 and between the drain electrode 16 and source electrode 18 to increase.

The programmable precision voltage source discussed above provides the voltages needed to properly electrically bias the IS-FET junction, by providing V_{source} to the source electrode 18, and V_{drain} to the drain electrode 16. The reference potential on the reference electrode 80, V_{ref}, is measured and used in a counter electrode excitation loop.

The counter electrode excitation loop continuously calculates the difference between V_{drain} and V_{ref}. This calculation defines the value of the gate voltage, V_{gate}. The gate voltage is a programmed value controllable by the user. An appropriate gate voltage reference is produced as V_{gate target} on the reference electrode 80 and compared to the measured gate voltage. The difference between the programmed voltage and the measured gate voltage is then applied to the counter electrode 14 as V_{counter}.

At least the gate voltage, counter voltage, and counter current are collected. The source current, I_{source}, and drain current, I_{drain}, may be measured based on these values. It will be appreciated that other measurements may be taken to ensure device health, for instance, to confirm that ISM 68 and/or CNT gating layer 34 are functioning correctly. To measure current, a differential voltage is measured across a fixed resistor value, and appropriate gain is applied to the signal to allow the ADC to sample it.

The current measurement transfer function is:

$${I}_{Drain or Source} = \frac{{V}_{ADC} * Vsource}{R * G * ( RANGEbit )} \\ {I}_{Counter} = \frac{{V}_{ADC} * Vsource}{R * G * ( RANGEbit )}$$

where V_{ADC} is the value of the raw ADC counts, V_{source} is the source voltage, R is the transimpedance resistance, G is gain, and RANGEbit is the bit range of the ADC. The transimpedance resistance is preferably about 1 kΩ to about 50 kΩ, and more preferably about 10 kΩ. G is preferably from about 1 to about 200, more preferably from about 2 to about 50, and even more preferably about 11. Finally, RANGEbit is preferably about (2¹²−1) to about (2³²−1), and more preferably about (2¹⁶−1).

The voltage measurement transfer function is:

$${V}_{measured} = {V}_{ADC} * \frac{Vsource}{( RANGEbit )}$$

where the values are as described above.

In one embodiment, the sensor system comprises a sensing platform for a continuous water resource monitoring by electrochemical detection and solution parameter correction. Continuous monitoring can be provided for drinking water, fresh water, wastewater, and water produced by reverse osmosis. This device may be used as a standalone sensor in environments where the water parameters (pH temperature, ionic strength) are controlled, or in concert with compensation sensors where water parameters are not controlled. Compensation sensors may include electrical conductivity, temperature, pH, oxidation reduction potential, and/or mass flow. Advantageously, the sensing system is particularly advantageous in low ionic strength environments (<100 mM).

It will be appreciated that the above sensors and methods allow for detection of nitrates at trace levels. For example, nitrates can be detected in water at levels preferably as low as about 10 ppm more preferably as low as about 100 ppm, and more preferably as low as about 100 ppm to about 1000 ppb.

Additional advantages of the various embodiments will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments but is not necessarily included. Thus, the present disclosure encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

### EXAMPLES

The following examples set forth methods in accordance with the disclosure. It is to be understood, however, that these examples are provided by way of illustration, and nothing therein should be taken as a limitation upon the overall scope.

### Example 1

### Fabrication of Ion-Selective Membrane Ink

A fresh stir bar was rinsed with about 5 mL of cyclohexanone, dried, and placed into a 100-mL plastic bottle, after which 45 milligrams of tridodecylmethylammonium nitrate (“TDMAN,” an ion exchanger sold under the name Tridodecylmethylammonium Nitrate Selectophore™, Sigma Aldrich, St. Louis, Mo.) were poured into the 100-mL plastic bottle with the stir bar. Next, 60 milligrams of 9-hexadecyl-1,7,11,17-tetraoxa-2,6,12,16-tetraazacycloeicosane (an ionophore sold under the name Nitrate Ionophore VI Selectophore™, Sigma Aldrich, St. Louis, Mo.) was weighed and poured into the same 100-mL plastic bottle. The 100-mL plastic bottle with stir bar was then placed onto a balance, and then 59.2275 grams of cyclohexanone (Sigma Aldrich, St. Louis, Mo.) were added to the 100-mL plastic bottle. The solution was then placed onto a hotplate with no heat, and stirring was set to 250 rpm. Once no particles were visible in the solution, the 100-mL plastic bottle was placed back onto the balance, and the weight was tared again, after which 495 milligrams of 2-nitrophenyl octyl ether (“NPOE,” Sigma Aldrich, St. Louis, Mo.) were added to the solution. The 100-mL plastic bottle was placed back onto stir pad and allowed to stir at 250 rpm for about 60 minutes. Finally, 172.5 milligrams of high-molecular-weight polyvinyl chloride powder (“PVC,” product number 81387, Sigma Aldrich, St. Louis, Mo.) was poured slowly into the stirring solution, and stirring was continued for 45 minutes at which point no particles were visible in the solution. At that time, the inkjet printable ion-selective membrane (“ISM”) was ready to print.

### Example 2

### Fabrication of Printable Carbon Nanotube Ink

A solution of 0.5% sodium dodecylbenzesulfonate (“SDBS,” Sigma Aldrich, St. Louis, Mo.) in DI water was prepared. Approximately 150 mg of multi-walled CNTs (Thin-Walled Carbon Nanotubes, manufactured by Cheap Tubes) was weighed and added to a beaker, and 400 mL of the 0.5% SDBS solution were added to the beaker, yielding a CNT stock solution having an optical density (OD) of approximately 8 at 550 nm.

The 0.5% SDBS solution was then used to clean a sonication probe before putting the probe into the beaker solution. The cleaned probe was placed inside the sonication box (a foam-lined box designed to reduce noise exposure during sonication) with the probe a few millimeters above the bottom of the beaker. Ice was added to a container to create an ice bath, and the beaker was placed inside this ice bath. The sonication box's doors were closed, and the gas valve was turned on to allow gas into the sonication horn. The control unit was set to 90% power for 20 minutes, and sonication was run twice, replacing the ice between the two 20-minute cycles of sonication.

After the two sonication cycles, the CNT solution was placed into a plastic container and transported for centrifugation. Two cycles of centrifugation were run at 14,000 rpm for 10 minutes each. Once complete, the fabricated stock solution was diluted to approximately OD=2.0 at 550 nm with 0.5% SDBS in DI water solution for spray-coating.

### Example 3

### IS-FET Nitrate Device Fabrication

First, 4.0-inch diameter and 0.18-inch thick substrates were cut from a Zeonor® ZF-188 film (Zeon Chemicals L.P, Louisville, Ky.) using a CO₂ laser. These wafers were then sputtered with 1,000 Å gold using a sputter deposition system (Model SC450, Semicore, Calif.) to form the source, drain, and counter electrode. This deposition was carried out by first placing the substrate on a magnetic stainless-steel holder, and a stencil mask (made from 150-μm thick molybdenum) was then placed on top of the substrate with neodymium magnets to affix the stencil mask and substrate to the stainless-steel holder. The Au target (0.125″ thickness×2.000″ diameter, purity: 99.99%) was mounted to the sputter head. The holder with the substrate and stencil mask was placed in the deposition chamber of the deposition system, and the pressure of the deposition chamber was decreased to 7.5×10⁻⁶ Torr. After the base pressure was reached, the system was run through an automated deposition procedure by first introducing argon (Ar) gas into the chamber until the pressure reached 2×10⁻² Torr, which is the plasma ignite pressure. The substrate holder was then rotated at 10 rpm, and DC power was ramped at a rate of 1 W/s up to the target of 50 W. After the power was maintained at 50 Watts for 15 seconds and the Ar gas was reduced to a deposition pressure of 2×10⁻³ Torr, the shutter was opened to start deposition. The pre-calibrated time for depositing 1,000 Å of Au was 4 minutes 20 seconds. After the deposition time expired, the shutter was closed, the power was reduced to 0 W at a rate of 2 W/s, and the Ar gas was turned off. The system then began the venting process to bring the chamber back to atmospheric pressure.

The substrate was then cleaned using isopropyl alcohol and alpha wipes before being dried in conveyor oven (Thermatrol® model NO-2410 from HIX® Corporation) set at 223° F. at 45.6 inches/minute. After drying, an encapsulation layer was applied by printing a solution of ZEONEX® 790R COP (Zeon Specialty Materials, San Jose, Calif.) in a 7-mm×11-mm rectangular shape onto the electrodes using an ATMA OE 67 screen-printer using a single durometer, shore hardness 70 squeegee and a stainless steel mesh screen. The flood bar was adjusted to yield a uniform flood across the mesh. Both squeegee and flood bar speeds were performed at 150 mm/s. The substrates were left to settle for 2 minutes to let any bubbles settle out of the ink. The ink was cured using the previously described conveyor oven at 256° F. at a speed of 45.6 inches/minute followed by UV light curing using a Heraeus DRS 10/12 UV oven. After the bubbles settled, the substrates were sent through the conveyor oven at 225° F., for one pass at 22.8″/minute, after which the substrates were passed through the UV oven for two passes.

Ultrasonic spray coating of a layer of the CNT dispersion from Example 2 was performed at 125° C. between the source and drain electrodes using a 100-mm freshly laser-cut polyethylene terephthalate (“PET”) stencil. Specifically, the spray coating involved first securing the substrates to a metal plate in an automated, programmable coating system (sold under the name ExactaCoat, Sono-Tek Corporation, Milton, N.Y.), and the deposition was performed at a pressure of 0.6 kPa. Once complete, the CNT-coated substrates were placed into a container of isopropanol (“IPA”) for 1 hour followed by placement in a container of DI water with stirring at 250 rpm for approximately 24 hours to ensure complete removal of the SDBS from the CNT material. After washing, the wafers were rinsed in IPA and heated at 55° C. on a hotplate for 15 minutes under atmospheric pressure.

After the substrates had dried, four layers of the ion-selective membrane dispersion from Example 1 were inkjet-printed so as to cover the source and drain electrodes as well the carbon nanotube layer. An inkjet printer (sold under the name CeraPrinter F-Serie by CERADROP, France) was used to perform this deposition. The temperature was set to 30° C. and after a solvent purge with cyclohexanone, the ISM dispersion was loaded into the printer. The substrate was then placed onto the platen stage with the electrodes in the direct center of the stage and the crosshairs aligned with the horizontal and vertical grids of the stage. Kapton® tape was cut and placed around the edge of the substrate until the perimeter of the substrate was completely encased with tape. The following parameters were used for the inkjet printing of the ISM ink: thickness of 0.18 mm; working distance of 0.16 inch; 45 μm splat diameter; frequency of 4500 Hz; four layers; 40% power; and print rate of 25 mm/s. The wafers were then cured in a vacuum oven (sold under the name Stable Temp® Model 282A by Cole-Parmer®, Vernon Hills, Ill.) at 30° C. and 100 mTorr overnight to fully remove the solvent residues. The devices were inspected with a microscope to confirm that the ISM film was correctly printed, covering both the source and drain electrodes.

### Example 4

### Step-Gate Response of TWCNT/SDBS Film

The CNT layer was the electrical transduction layer of the device, where the phase boundary potential of the ISM film is converted to a change in CNT resistance, likely due to changes in the oxidation state of the CNT material. To evaluate the step-gating performance, a step-voltage gating test was carried out to test the CNT film.

An IS-FET nitrate device was fabricated as described in Example 3 except without formation of an ISM layer. A step-voltage gating test was performed using a CNC robot system (sold under the name Shapeoko XXL, by Carbide 3d, Torrance, Calif.) to automatically move the sensors to various vials of the ion strength adjustor solution. A 1.0 mM KNO₃ solution with 0.1% solution of (NH₄)₂SO₄ (Cole-Parmer®, Gardena, Calif.) was used as an ion strength adjustor solution. The devices were swept back and forth from 200 mV to 600 mV versus an external Ag/AgCl (supersaturated KCl) reference electrode in 50 mV steps every 20 minutes, the first upward half cycle being illustrated in the left side of FIG. 8. A custom nitrate PCB with the components shown in FIG. 6 used a precision voltage reference to control the gate voltage applied.

The response of the SDBS CNT-based IS-FET sensor was logarithmically related to the nitrate concentration, following the Nernstian behavior (FIG. 8). The measured drain current with 50 mV applied is shown on the left. The cycle stability of the CNT film gate response from 200-600 mV vs. an Ag/AgCl (supersaturated KCl) reference electrode (on right in FIG. 8).

### Example 5

### Sensitivity Test of IS-FET Nitrate Sensor

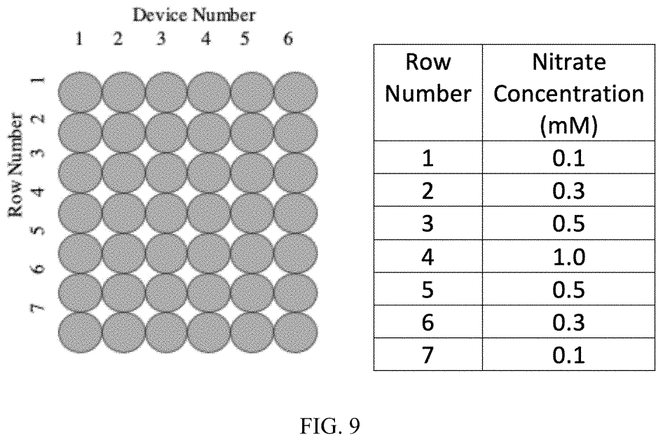

To evaluate the sensitivity of IS-FET nitrate devices fabricated in Example 3, a matrix of solutions with varying nitrate concentrations was assembled as shown in FIG. 9. Each column represents a unique nitrate sensing device, and each row is representative of a specific concentration of nitrate solution. The nitrate source was KNO₃ and, as shown in FIG. 9, the concentration of KNO₃ in the testing solutions varied from of 0.1 mM to 1.0 mM. All solutions contained 0.1% (by volume) ammonium sulfate as an ion strength adjuster to simulate the ionic conductivity to the natural water. Each circle represents approximately 5 mL of the nitrate solution.

The IS-FET nitrate sensing devices were presoaked in the first row of nitrate solution for two hours under a constant applied potential of 330 mV vs. the supersaturated KCl reference electrode. Once the presoaking process was completed, the devices were moved to a new solution in the next row and held for 20 minutes. This process was repeated until a 10-cycle test was performed in each of the seven solutions. A 50 mV voltage difference between the source and drain electrode was built into a PCB board with the circuitry as shown in FIG. 6 to measure the resistance of the CNT film, which was the output signal. The IS-FET nitrate sensing devices were connected to respective PCBs, which supplied the electrical excitations as well as recorded the output data into a CSV file inside a connected desktop PC. When applying a gate potential of 330 mV vs. the reference electrode, the source, drain, and counter current were recorded by the PCBs.

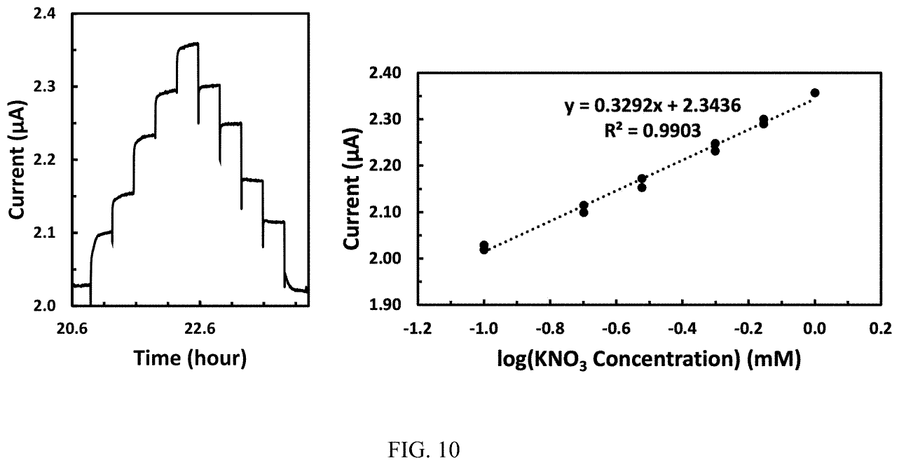

FIG. 10 (left) shows the drain current (Id) versus time of the sensitivity experiment for the IS-FET nitrate sensor fabricated as described in Example 3. Each step represents a solution with different nitrate concentration. FIG. 10 (right) shows the plot of average Id of each step versus the logarithm of nitrate concentration to understand the hysteresis of the sensing device. This linear response of the Id vs. log[NO₃] demonstrated that the signal response of the IS-FET nitrate device followed Nernstian behavior.

### Example 6

### Selectivity Test of IS-FET Nitrate Sensor

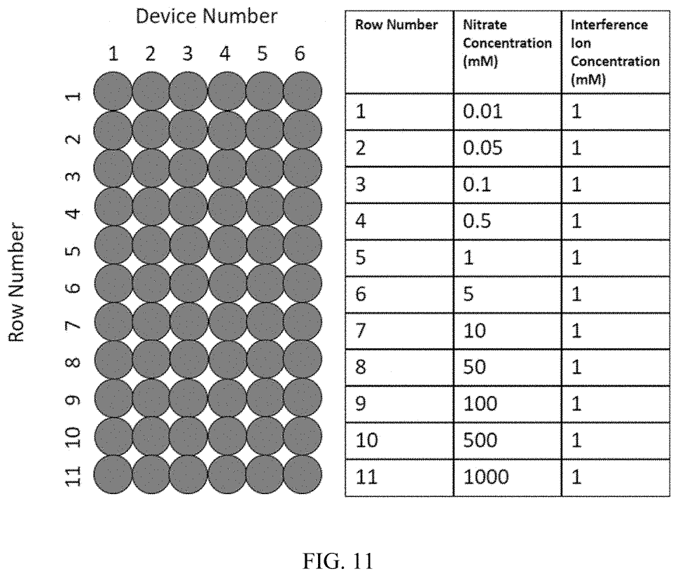

Selectivity is an important criterion to evaluate the IS-FET nitrate device performance for commercial applications. A matrix of 5-mL plastic vials was prepared on the table in array such that each of the eleven rows represented a concentration of nitrate, and each column represented a unique device. All solutions contained a constant level of interference ion and varied nitrate (i.e., primary) ion concentrations of 0.01, 0.05, 0.1, 0.5, 1.0, 5.0, 10, 50, 100, 500, 1000 mM KNO₃ (the row number in FIG. 11) with 1 mM of the particular interfering ion. That is, the interfering ion in each vial was different by column, with the interfering ions Br⁻, Cl⁻, I⁻, ClO₄⁻, H₂PO₄⁻, or SO₄²⁻ being in columns 1-6, respectively (FIG. 11).

The devices fabricated as in Example 3 were presoaked in the first row of nitrate solution for two hours at a constant gate potential of 330 mV vs. Ag/AgCl. The devices were then moved to a new row of solutions every 20 minutes. The nitrate devices were connected to PCBs that supplied the electrical excitations as well as recorded output data, which was transferred to a CSV file on a connected desktop PC (similar to that described in Example 5).

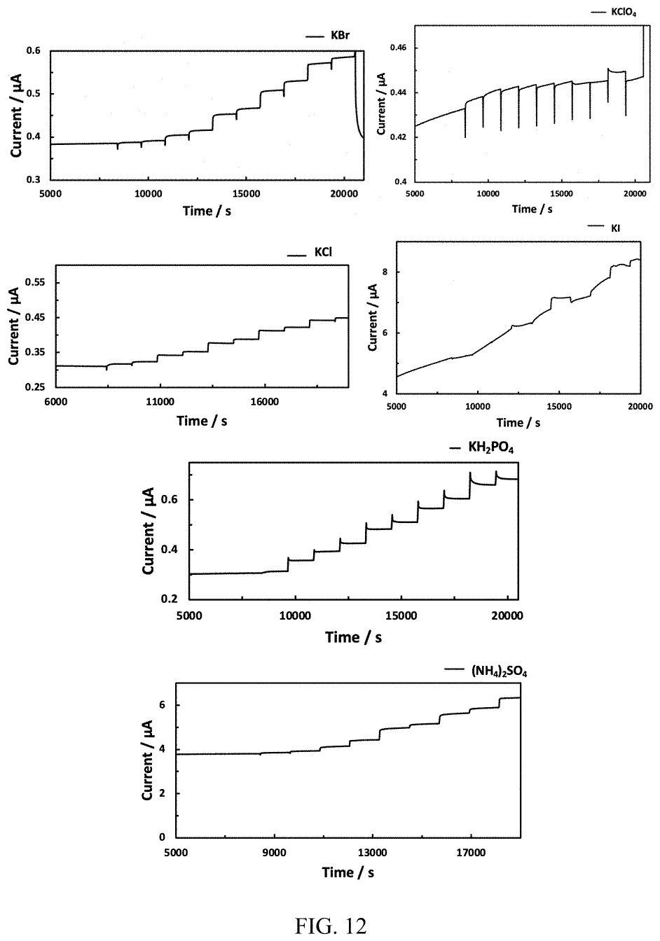

FIG. 12 shows the step response in the selectivity tests using Br⁻, Cl⁻, I⁻, ClO₄⁻, H₂PO₄⁻, and SO₄²⁻ as the interfering ions, along with the source used to supply each interfering ion. Generally, the step response in the selectivity test was more pronounced, indicating the ISM was not selective to these ions. As shown in FIG. 12, a step response of current was present in the presence of Br⁻ contamination, indicating that Br⁻ did not interfere with the nitrate device. Low interference was observed by Cl⁻, H₂PO₄⁻, and SO₄²⁻ ions. The step response was diminished in the presence of ClO₄⁻ and also in the presence of I⁻, indicating strong interference by ClO₄⁻ and I⁻.

### Example 7

### Long-Term Drifting Test of IS-FET Nitrate Sensor

A long-term drifting test was carried out to evaluate the stability of the nitrate sensor over a practical period of time. For this testing, 30 nitrate sensing devices were fabricated as described in Example 3, but with 2 ISM layers on 10 of the devices, 4 ISM layers on 10 other of the devices, and 10 ISM layers on the remaining 10 devices. Each device was placed in its own small plastic vial with 20 mL of 1.0 mM KNO₃+0.1% ISA solution. Because the drifting test would be carried out over about a month, the vial lids were fabricated so that the reference electrode and nitrate device were sealed in the lid, and Teflon® tape was used to seal the lid and vial to prevent water evaporation.

The nitrate devices were inserted into PCBs with the circuitry shown in FIG. 6 that were connected via male and female ribbon cables to the driver circuit for electrochemical testing. An uninterrupted power supply was used to connect to the drifting test system so that no power interruptions would affect the extended experiment. The test involved applying a constant gate potential of 330 mV vs. Ag/AgCl, and the current was measured every 5 minutes. The sensing signal was collected by the PCB board and was interpreted by a Python program on a connected desktop PC.

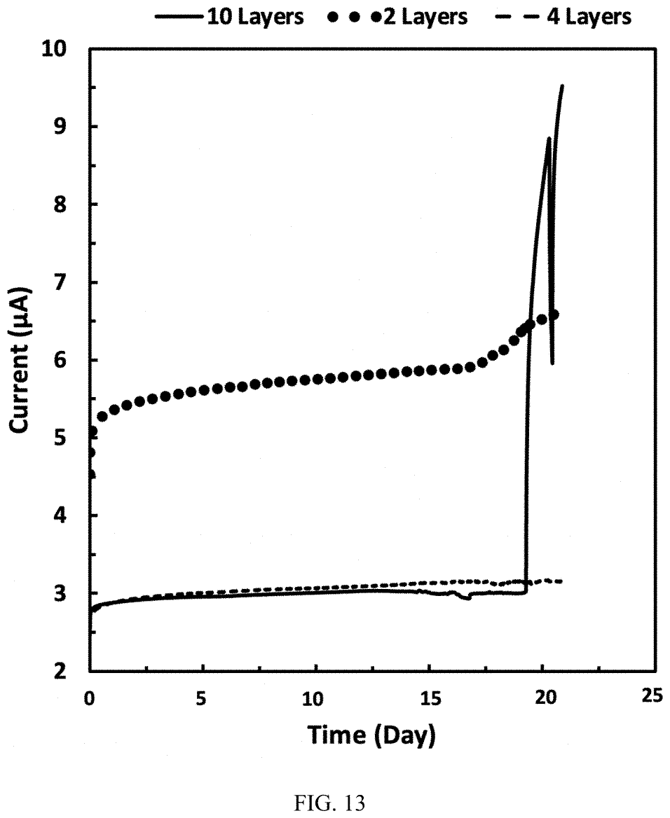

FIG. 13 shows the drain current with respect to time of the different nitrate devices. Ideally, the drain current of a nitrate device with excellent stability should stay the same with no current loss or increase under the constant electrochemical testing condition. Therefore, the flat curve of drain current indicates long-term stability of the nitrate device. In particular, when subjected to a Drifting Test as described in this Example, the nitrate device should experience a current change (either loss or increase) of no more than about 1.5 μA, preferably no more than about 1 μA, and even more preferably no more than about 0.5 μA. These Drifting Test results are preferably obtained at a time period of at least about 10 days, preferably at least about 15 days, more preferably at least about 20 days, and even more preferably at least about 24 days.

We claim:

1. A sensor comprising:
a substrate;
a source electrode on said substrate;
a drain electrode on said substrate;
a carbon nanotube gating layer connecting said source electrode and said drain electrode;
an ion selective membrane on said carbon nanotube gating layer, wherein said ion selective membrane comprises:
a polymer, an epoxyacrylate oligomer, or both a polymer and an epoxyacrylate oligomer, said polymer being chosen from polyvinyl chloride, polyacrylate, polymethacrylate, or combinations thereof;
an ionophore chosen from cyanoaqua-cobyrinic acid heptakis(2-phenylethyl ester), 1,6,10,15-tetraoxa-2,5,11,14-tetraaza-cyclooctodecane, 1,7,11,17-tetraoxa-2,6,12,16-tetraazacycloe-icosane, 9,11,20,22-tetrahydrotetrabenzo[d.f,k,m] [1,3,8,10]tetra-azacyclotetradecine-10,21-dithione, 9-hexadecyl-1,7,11,17-tetraoxa-2,6,12,16-tetraazacycloeicosane, or combinations thereof;
an ion exchanger chosen from tridodecylmethyl ammonium nitrate, tetradodecyl ammonium nitrate, tetraoctylammonium nitrate, potassium tetrakis(4-chlorophenyl) borate, tetrakis(4-chlorophenyl)borate tetradodecylammonium salt, or combinations thereof; and
a plasticizer; and
a counter electrode on said substrate, there being no direct physical contact between said counter electrode and any of said source electrode, drain electrode, carbon nanotube gating layer, or ion selective membrane.

2. The sensor of claim 1, wherein said plasticizer is chosen from 2-nitrophenyl octyl ether, dibutyl phthalate, bis(2-ethylhexyl) sebacate, bis(2-ethylhexyl) phthalate, or combinations thereof.

3. The sensor of claim 1, wherein:
said source electrode comprises a source working end and a source lead end; and
said drain electrode comprises a drain working end and a drain lead end, wherein said carbon nanotube gating layer connects said source electrode and said drain electrode at said drain working end and source working end.

4. The sensor of claim 3, wherein:
said source electrode further comprises, at said source working end:
a first source electrode sidewall facing generally away from said drain electrode;
a second source electrode sidewall facing generally towards said drain electrode; and
an upper source electrode surface extending between said first source electrode sidewall and said second source electrode sidewall;
said drain electrode further comprises, at said drain working end:
a first drain electrode sidewall facing generally toward said source electrode;
a second drain electrode sidewall facing generally away from said source electrode; and
an upper drain electrode surface extending between said first drain electrode sidewall and said second drain electrode sidewall; and
said first drain electrode sidewall and second source electrode sidewall are spaced apart such that a first exposed substrate portion is created therebetween, and said carbon nanotube gating layer is in contact with said upper source electrode surface, said second source electrode sidewall, said first exposed substrate portion, said first drain electrode sidewall, and said upper drain electrode surface.

5. The sensor of claim 1, wherein said ion selective membrane entirely covers and encompasses said carbon nanotube gating layer.

6. The sensor of claim 5, wherein said ion selective membrane further covers:
said first source electrode sidewall;
any portion of upper source electrode surface not covered by the carbon nanotube gating layer;
any portion of upper drain electrode surface not covered by the carbon nanotube gating layer; and
said second drain electrode sidewall.

7. The sensor of claim 3, wherein:
said counter electrode comprises a counter working end and a counter lead end; and
said sensor further comprises an encapsulant layer covering:
said counter electrode intermediate said counter working end and said counter lead end;
said source electrode intermediate said source working end and said source lead end; and
said drain electrode intermediate said drain working end and said drain lead end.

8. The sensor of claim 7, wherein said encapsulant layer is not on said counter working end, said source working end, or said drain working end.

9. The sensor of claim 8, wherein said ion selective membrane extends from at least said encapsulant layer and fully covers and encompasses said source electrode at said source working end and said drain electrode at said drain working end.

10. The sensor of claim 7, wherein said encapsulant layer is not on said counter lead end, said source lead end, or said drain lead end.

11. The sensor of claim 7, wherein said encapsulant layer has a resistance of at least about 1 MΩ.

12. The sensor of claim 7, wherein said encapsulant layer is formed from a composition comprising a polymer chosen from cyclic olefin polymers, fluorinated polymers, tetrafluoroethylene and hexafluoropropylene copolymers, polyvinylidene fluoride, polyether ether ketone, polyetherimide polyphenylene sulfide, polysulfones, polyoxymethylene, polyimides, polyamides, polyether sulfones, polyethylene terephthalate, polyacrylates, polymethacrylates, polystyrenes, polyesters, polyethylene naphthalate, polysilicones, or combinations of the foregoing.

13. The sensor of claim 4, said first drain electrode sidewall and second source electrode sidewall are spaced apart at a distance D1, where D1 is about 100 μm to about 1 cm.

14. The sensor of claim 3, wherein:
(i) said source electrode at said source working end has a width of about 200 μm to about 2 cm;
(ii) said drain electrode at said drain working end has a width of about 200 μm to about 2 cm; or
(iii) both (i) and (ii).

15. The sensor of claim 1, wherein the carbon nanotube gating layer comprises metallic carbon nanotubes.

16. The sensor of claim 1, further comprising a second sensor formed on a substrate, wherein said second sensor is selected from the group consisting of an electrical conductivity sensor, temperature sensor, pH sensor, oxidation reduction potential sensor, or a combination thereof.

17. A sensing device comprising a sensor according to claim 1 further comprising a reference electrode and a power source, wherein the reference electrode and power source are connected to the sensor.

18. A method of monitoring for the presence of an analyte in water, wherein said method comprises contacting a sensor according to claim 1 with water to be monitored.

19. The method of claim 18, wherein said analyte is a nitrate.

20. The method of claim 18, wherein said sensor is capable of detecting nitrate present in water at levels as low as about 10 ppm.

21. The method of claim 18, wherein said contacting comprises positioning said sensor within a flow path of the water to be monitored.

22. The method of claim 18, wherein said sensor is connected to a reference electrode and a power supply.

23. The method of claim 18, wherein an electrical property of the CNT gating layer varies in response to the presence of said analyte.

24. The method of claim 23, wherein said electrical property is impedance.
